(19) United States
(12) Patent Application Publication
(10) Pub. No.: US 2024/0053318 A1
(43) Pub. Date: Feb. 15, 2024

(54) SYSTEMS AND DEVICES FOR TREATMENT BY MEANS OF TRANSFER OF INFORMATION IN WATER

(71) Applicant: Holger KIEWE GENANNT KÖNIG BERTHOLD, PAZZALLO (CH)

(72) Inventor: Holger KIEWE GENANNT KÖNIG BERTHOLD, PAZZALLO (CH)

(21) Appl. No.: 17/801,164

(22) PCT Filed: Mar. 17, 2022
(86) PCT No.: PCT/EP2022/056959

§ 371 Date: Aug. 19, 2022

(30) Foreign Application Priority Data
Mar. 17, 2021 (US) 63162085
May 4, 2021 (US) 63183653

(51) Int. Cl. G01N 33/18; C12Q 1/70; G16H 40/63

(52) U.S. Cl. CPC G01N 33/1826; C12Q 1/70; G01N 33/1886; G16H 40/63

(57) ABSTRACT

A system and method/apparatus are provided which involves the transfer of beneficial information as well as beneficial substances through the water into living organisms which consume the water. This allows for the treatment of living organisms en mass using pre-existing or dedicated piping as a distribution system. In addition, authorities are provided with the ability to mete out beneficial treatments, in some cases using beneficial substances carried in the water lines, on an as needed basis, according to a treatment protocol that the authorities believe to be most effective. In an embodiment, cabins are provided that allow a user to be treated and identified such that a treatment certificate can be issued on the spot at a place of need, such as at an airport.

### CROSS REFERENCE TO RELATED APPLICATIONS

This application claims the priority of U.S. Provisional Application No. 63/162,085, filed 17, Mar. 2021 and U.S. Provisional Application 63/183,653, filed 4. May 2021.

### BACKGROUND OF THE INVENTION

This invention relates to systems, devices and apparatuses enabling treatment methods using water as a communication means. Water is an essential component of life and bears beneficial substances like minerals. It is known that water can be treated with various substances such as chlorine, fluoride, magnesium, for example, using a water treatment plant which converts sewage into clean, potable water. Oral vaccination against Typhoid for example, is also known. The human body is composed of at least 80% water and so the nature of water entering the body can have an influence on the living organism's health. The unique characteristics of water are well documented. Still further, it has already been proven that certain water can more easily enter the cell structure of a living organism and have an effect on the living organism. One company that promotes this concept is Plocher of Germany (see plocher.de).

### SUMMARY OF THE INVENTION

The embodiments described below describe methods of treating living organisms on a mass scale using public water utility services.

A system and method/apparatus are provided which involve the transfer of beneficial information as well as beneficial substances through the water into living organisms which consume the water.

An object of the invention is to treat living organisms en mass using pre-existing or dedicated piping as a distribution system.

Another object of the invention is to provide authorities with systems, devices and apparatuses for meting out beneficial treatments, in some cases using beneficial substances carried in the water lines, on an as needed basis, according to a treatment protocol that the authorities believe to be most effective.

Still another object of the invention is providing cabins that allow a user to be treated and identified such that a treatment certificate can be issued on the spot at a place of need, such as at an airport.

Other objects will be apparent to the reader as implied by the instant disclosure.

### BRIEF DESCRIPTION OF THE DRAWINGS

The attached drawings represent, by way of example, different embodiments of the subject of the invention.

FIG. 1 is a schematic, cross-sectional view of an accumulator of the present invention.

FIG. 2 is a schematic view of a distribution system for vaccinations of the present invention.

FIG. 3 is a schematic, cross-sectional view of a refrigerated carrier line including four different vaccination rings.

FIG. 4 is a schematic, cross-sectional view of a refrigerated carrier line enclosing four tubes, three of which are filed with different vaccinations and one is used as a transport tube.

FIG. 5 shows a flow chart of a process of early detection and protection according to an embodiment.

FIG. 6 shows a flow chart of a process of hotel Corona monitoring and finding a sick guest according to an embodiment.

Those skilled in the art will appreciate that elements in the Figures are illustrated for simplicity and clarity and have not necessarily been drawn to scale. For example, dimensions may be exaggerated relative to other elements to help improve understanding of the invention and its embodiments. Furthermore, when the terms ‘first’, ‘second’, and the like are used herein, their use is intended for distinguishing between similar elements and not necessarily for describing a sequential or chronological order. Moreover, relative terms like ‘front’, ‘back’, ‘top’ and ‘bottom’, and the like in the description and/or in the claims are not necessarily used for describing exclusive relative position. Those skilled in the an will therefore understand that such terms may be interchangeable with other terms, and that the embodiments described herein are capable of operating in other orientations than those explicitly illustrated or otherwise described.

### DETAILED DESCRIPTION OF THE PREFERRED EMBODIMENT

The following description is not intended to limit the scope of the invention in any way as it is exemplary in nature, serving to describe the best mode of the invention known to the inventor as of the filing date hereof. Consequently, changes may be made in the arrangement and/or function of any of the elements described in the exemplary embodiments disclosed herein without departing from the spirit and scope of the invention.

The system and the method according to this invention concerns systems, devices and apparatuses that facilitate the treatment of living organisms, such u their vaccination against disease, by means of information encoded in water and the transfer of said information into and through water.

### Two Types of Distribution System:

Distribution system of type A: Inoculation by means of encoded water in public water pipeline systems, transferring the water either to buildings (such as offices, domestic homes, etc.) or to a dedicated place of collection.

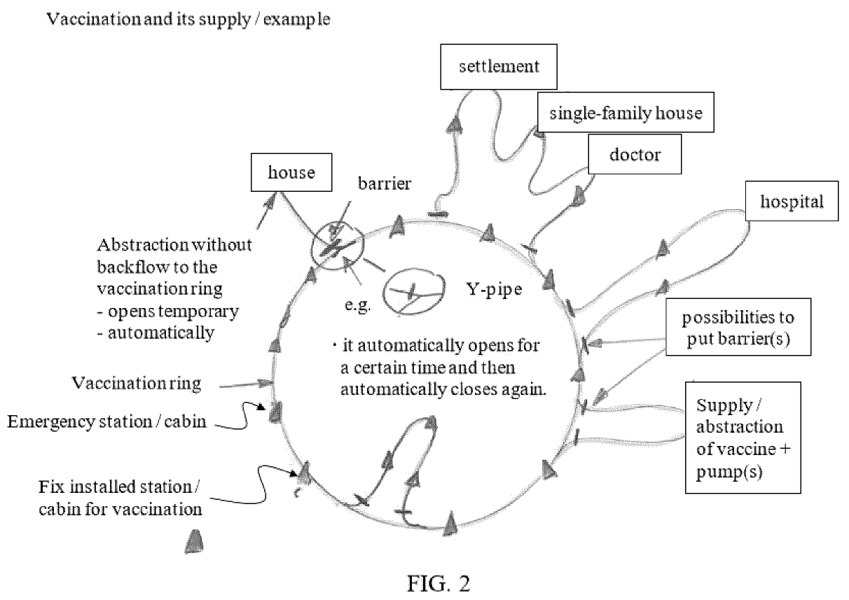

Distribution system of type B, schematically shown in FIG. 2: Inoculation by means of encoded water in dedicated water pipeline systems, transferring the water either to buildings (such as offices, domestic homes, etc.) or to a dedicated place of collection.

### 1. Embodiment

Information is implemented into water by means of encoding the water. This is realized without inducing impurities (such as substances) into the water.

The implementation of the information into water can be accomplished directly or by means of a carrier such as balls (preferably small balls) or rings made of materials impregnated with the information. Balls are selected because they minimize the back pressure caused by the drag of the water over the balls. Rings are selected when the ring is held securely in the pipe so as to allow the water to flow through the center of the ring. Such carriers allow to transfer the information in a quick and intense manner into the water, because, due to their large surface area or orientation with respect to water flow, they promote and/or catalyse the transfer of information into the water.

In one version, the carriers, such as the rings mentioned above, remain static in a distribution system while the water flushes around the carriers.

Alternatively, the carriers are directly added to a distribution system at one location and then, after the information is transferred into the water and memorized by the water, taken out of the distribution system by means of for example, filtering. In another alternative, the information is transferred into the water by means of the inside walls of the piping of a distribution system, or by means of a statically installed catalyser made of a suitable substance such as nanomaterials or metallic grid. The static installation of a catalyser may be inside a distribution system or in a water tank/basin. The water flushes around these manna and receives thereby the information by contact. If the information is directly encoded on the walls of the piping of a distribution system, the information is transferred into the water while the water flows along these inside walls. The carriers as mentioned in the instant paragraph are reusable.

One can easily imagine how on a simple basis, information can be transferred into water. In one example, an induction coil can input heat into the water stream which a sensor downstream can sense in order to determine the frequency of the heat input into the stream, this input frequency corresponds to a mouse code message. In another example, sound can be input and carried in the water, thereby carrying information, even a message. Electromagnetic waves can be transmitted by the water, thereby carrying a radio signal bearing a message. Water can also be magnetized in pulses in one location which can be read downstream so as to carry information. Light pulses can be transmitted by water in pipes, even pipes that include comas where the corners include mirrors reflecting the light down the next pipe. Colors or contaminates, or substances can be intermittently injected into water, whose later detection downstream is capable of carrying a signal.

It has also been found that water has memory and so can carry information downstream by means of transfer of said information from carriers, such as balls (preferably small balls) or rings to the water, using an orgone accumulator.

It has already been proven that certain water can more easily enter the cell structure and have an effect there. With this knowledge and in combination with a suitable vaccine, whose information or reaction is reflected in the body, the body can be strengthened and a stronger (or weaker as required) defence reaction can occur.

In addition, the article “Kurzer Prozess im Wassercluster” (https//www.pro-physik.de/nachrichten/kurzer-prozess-im-wassercluster) and the references mentioned therein provide proof of the influence of types of water on the immune system, cells, blood, etc.

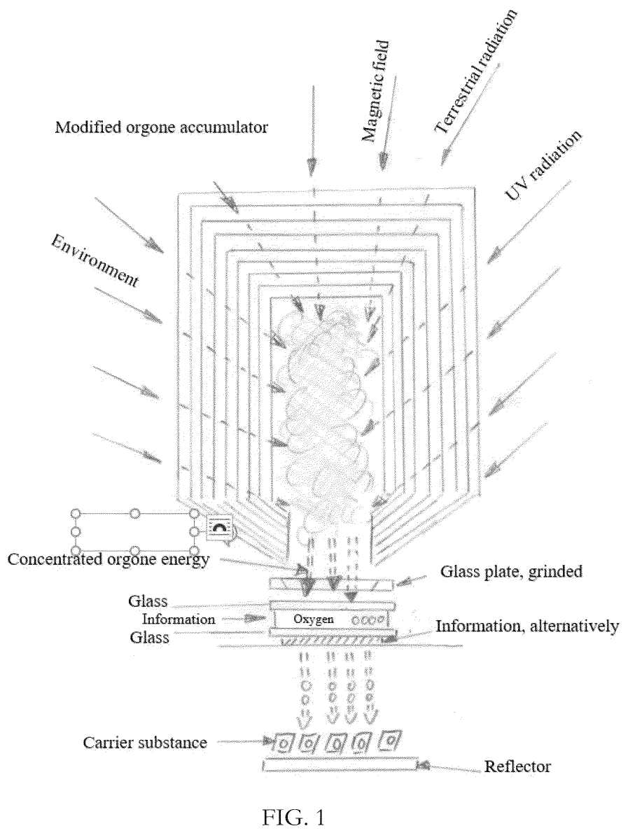

Referring now to FIG. 1, showing an orgone accumulator (this name origins from Mr. Wilhelm Reich) absorbs cosmic radiation or vibration and amplifies it. The wall of the orgone accumulator amplifies the cosmic radiation. Depending on the number of layers of the orgone accumulator, its power increases, the radiation becomes stronger in the orgone accumulator. The radiation is diverted to one side from the orgone accumulator and finally hits the information carrier, which is to be copied, so that its information is transferred and stored on the carrier/carrier body etc. This carrier/carrier body (also quartz sand etc.) can then deliver its information e.g. to water, but also to other things or living beings (not only to the water). A reflector is attached underneath the carrier material, which throws the radiation back again on the carrier/carrier body and thereby strengthens the input. This carrier gives the injected information to the water.

Water stores all substances and information. Water has this property and this is scientifically proven and known. The water has a kind of memory.

Earth magnetic field/radiation is indicated on FIG. 1 with arrows: The transferred information and the carrier substance are subjected to a magnetic field/radiation, which corresponds to the magnetic field of the earth long time ago. The information carrier is subjected partly during, before and if necessary afterwards to this magnetic field. Also, the carrier/carrier body is subjected partly during, if necessary before and if necessary after this magnetic field. Sometimes all three must be applied, so that the goal is reached.

This combination by the orgone accumulator and the magnetic field/vibrations, makes possible a strengthened reaction to biomass, the water, the organism of each individual cell of animals and humans, all living beings. These oscillations are entered on the carrier/carrier body (solid, gas, water, blood, etc.).

This combination is not known in the prior art, it has been discovered by the applicant and is typically very important for the success of the treatment.

This combination of the orgone accumulator in connection with the magnetic field/radiation treatment does not exist in the prior art and protection is sought by the present patent application.

As a matter of background information, evidence of the presence of cosmic radiation was established decades ago. This radiation is ultimately also strengthened by the Earth's magnetic field, which surrounds the Earth, as well u on the Earth prevails and acts on all life forms, plants as well as rocks etc. The moon phases, in particular 5 days before and 5 days after full moon, strengthen with their attraction this cosmic radiation and/or the effect on living beings on Earth. The magnetic field on the Earth, which affects us humans, changes in these 11 days, it becomes or is stronger.

This strengthening influences water and all life forms whose composition is primarily that of water.

The radiation or waves ultimately penetrate all substances and influence human beings. Importantly, it reaches the water in our cells.

Even the stock exchange has knowledge of it or uses this knowledge. A study has shown that the full moon has an influence on the stock exchange prices or stock exchange fluctuations. The cosmic radiation is strengthened by the magnetic field, as discussed in the article available at the following link: www.tagesschau.de/wirtschaft/boerse/hr-boerse-story-15673.html.

The magnetic field influences, among other things, the growth of plants.

The cosmic radiation in connection with the magnetic field makes the water react in the plants, animals and in the human body. This water is ultimately a transmitter.

In experiments, plants, humans, animals or the water reacted directly to cosmic radiation. In every living thing there is water and there are inanimate objects such as lakes, water bodies, etc. which also react to cosmic radiation.

The water is our key component. Be it steam, mist, liquid or solid water, water reacts in our body and as a result, we either get sick or healthy or change etc.

Media capable of storing information (such as in a lattice) and as a transmitter of this information to a third medium, such as water include: Quartz sand, quartz rock, quartz flour and various other rocks.

Quartz balls or rings. Steel, stainless steel, which has been treated on the surface.

Plastics, which were treated at the surface (among other things surface enlargement).

There is already research on this, which has shown that water, as well as sand, forms/draws patterns and structures in the sand at different frequencies introduced, as well as in the water.

The water shows with its oscillations an almost 1:1 image of a human cell or of the cells of other animals. This information, that for example a drop of water divides itself at certain oscillations into approx. compartments like a caterpillar or millipede etc., this information is in the water.

The water has stored various forms and structures of animals etc. in itself. Via oscillation (certain frequencies), which one introduces on the water, their forms are to be recognized clearly.

Plants have water in all branches. We humans have water in every cell and consist of 70%-80% water.

Aluminum foil, silver foil, quartz powder or sand, but also other substances serve preferably as carriers of a copy of the information.

Devices can be useful to harness this energy, such as orgone energy, a pseudoscientific concept variously described as an esoteric energy or hypothetical universal life force. Orgone energy arises through cosmic orgone energy or cosmic radiation. And is a concentrated orgone energy/cosmic radiation, which penetrates everything as before.

However, this radiation/vibration can be so intense that the carrier absorbs and stores this vibration and e.g. transferring it to the water.

Storage of cosmic energy whose oscillations (which were stored intensively in connection with the previous additional magnetic treatment) are continuously given off over several years to a third medium like e.g. water.

The yield of plants (grain, corn, etc.) after being treated using systems, devices and apparatuses of the invention can be increased by up to 100% above a normal yield.

Transferring Information into Tubes:

Own production of a color, which was added only with quartz sand or special plastic.

This sand or foreign materials, such as plastics, carry the information, sometimes the paint is directly treated with the particular information input to reinforce this information. The water then receives the information when it flows over this paint.

Information on Grid/Metal:

Depending on the information, only a grid or metal is suitable to receive the information inputs, as otherwise the carrier/carrier body.

If necessary, this information can be amplified by magnetism (magnets on the wire) or a current field. Also, the grid can be painted or varnished with the treated paint, which also received the information.

Regarding the interaction with a sphere or ring, this is a transmission of cosmic vibrations, information that has bear introduced into the carrier. Quartz sand transmits especially well. This may possibly have to do with a possible charging of the crystalline structure in the sand. As with a quartz clock, the quartz stone provides the cadence or vibration. Therefore, the quartz sand, stone or powder in whatever form stores the information better and faster by static charging and for transmission or information transfer into the water. Also, magnetic treatment is in the end an amplification of the entry of the information on the carrier, but also the release of information into the water is strengthened by it.

How do the water molecules interact? It can be only by the vibration of the input or by the magnetic field. Thus it would be by means of the frequency and the field strength.

### 2. Embodiment

A vaccine, optionally an external vaccine, is added to the water by means of impurities injected into the water or mixed therein.

### 3. Embodiment

Oral vaccination may be administered via water lines by dissolving a substance into the water or adding the substance as a particulate. For example, Typhoid or Polio can be administered in this manner.

### 4. Embodiment

When water comprising information as described above is consumed by a consumer, an information transfer process referred to as Coulomb decay or the Auger effect takes place such that the corresponding properties of the water are transferred to the consumer. The realization/encoding of such water is accomplished directly through and at the water supply companies and/or other companies, and may also be done at intermediate sections/locations in a distribution system.

### Applications:

### At Sewage Plats:

Information may be transferred into the water at a sewage plant due to a stronger/more intense regeneration being possible given that purification will take place prior to the water being released into the conductive piping. The information may also be added to the water at the end of a cleaning process as typically performed at a sewage plant. Once the water leaves the sewage plant, the information in the water may also be encoded so as to have a positive impact on the environment such as rivers, lakes or the like. For example, the water may be encoded to benefit living organisms such as aquatic life both plants and animals living in runoff estuaries, rivers, lakes and oceans.

### Surveillance of Sewage Water

Shortened reaction time to consumer needs, such as public health needs, is possible thanks to the invention's automated surveillance of sewage water at sewage plants, optionally, direct transmission to authorities, government agencies, health authorities, institution, water supply companies, etc., such to quickly allow application of countermeasures.

In one embodiment, the invention makes possible the detection of infectious agents such as microbes, bacteria and viruses in the water. The sewage water may be monitored using water testing means (automated sampling and detection) for particular infectious agents or contaminants, including the detection of their type(nature/variety and their density of presence. In another embodiment, the detected infectious agents or contaminants are reported to a central processor adapted to map the detections on a map, thereby giving authorities an almost immediate, real-time indication of hot spots and regions likely to become super spreaders. Detection and/or mapping of the affected regions saves time as infectious agents (such as Corona viruses) may be detected in sewage water although diseased individuals don't show symptoms of the corresponding disease.

The method of the invention may be implemented in software instructions readable by a computer processor which executes the instructions for detection and mapping. Realization of a specific computer implemented program which automatically transmits data regarding the detection and/or mapping to authorities, government agencies, health authorities, institutions, and/or water supply companies, etc. The data may be analyzed by the same or another computer implemented program in order to calculate and/or compile, optionally in a fully automated manner, a corresponding treatment response.

### Administration/Application

Concerning the first embodiment in conjunction, for example, with distribution system of type A, the water with the therein implemented information (encoded as described above) is transferred by means of public water pipeline systems to buildings (such as offices, domestic homes, etc.). In doing so, it is less likely to over-administer the treatment response possible to vaccinate with an overdose because only the information implemented into the water is transferred to a consumer of the water. The information as transferred to the consumer initiates a reaction in the consumer's body.

Via administration of the water through contact with the mucosa, derma, or epiderma, alternatively by means of drinking the water, the beneficial treatment is achieved.

Concerning the second embodiment in conjunction, for example, with distribution system of type A, places of collection may be centralized places at which water comprising the therein implemented information (encoded as described above) is made available to residents or other institutions.

Concerning the third embodiment in conjunction, for example, with distribution system of type A, when bottling of the water into bottles or other types of containers, these bottles or other types of containers may then be sold/distributed on the market, for example, by the use of a beverage distribution system.

### Encoded Water as Described in the Instant Description:

The applicant has discovered that water has a memory which allows the detection of every substance that has ever been added to the water even after, for example, a one to a million dilution. The applicant has learned that water does not only memorize information, but also reacts with the therein implemented information so as to deploy a corresponding effect and accordingly transfer it to consumer's body. The human body consists of typically 80% water. All human organs, the human's tissue, etc. are flushed and interfused by the water.

The invention allows an administration of a vaccine without the need of a syringe in order to inject the vaccine, for example, into the bloodstream. The typhoid/oral vaccination is known and has been administered for decades. The administration of water with therein implemented information by means of a distribution systems of type A or type B (as described above) is faster and more cost-efficient that the distribution means of the prior art. On the basis of data from the surveillance a of sewage water at sewage plants, an inoculation may be administered to human bodies within a very short time, typically within a few days. The administration can be done either directly into buildings (such as offices, domestic horns, etc.) or at dedicated places of distribution.

### Mixing of the Water:

Concerning embodiment 1: There is no need for mixing as the water has the corresponding information implemented therein. A subsequent rinsing of the distribution system may be performed. However, it should be noted that such a rinsing is optional.

Concerning embodiment 2: The information may be implemented into the water at several different locations/stations of the distribution system.

Also, a pre-mixed vaccine, without the need to be diluted, may be admixed directly, one to one, to the water in the distribution system, such that it can be distributed to, for example, buildings (such as offices, domestic homes, etc.) or a dedicated place of collection. For a neutralization of the vaccine (the information implemented in the water) in the piping of the distribution system, it would be sufficient to simply rinse these piping with water or, alternatively, a corresponding agent is rinsed though the piping once the inoculation is completed.

Further, a dedicated water pipeline system may be realized, allowing for transport of the vaccine to buildings (such as offices, domestic homes, etc.) or to a dedicated place of collection.

Concerning embodiment 3: A ready-to-drink mixture or a mixture for dilution.

### A Diseased Human and the Detection in Water:

The water in a human body of a diseased individual comprises inevitably different markers as the one of a healthy individual. Consequently, diseases may also be detected though analysis of the water in a human body.

### The Human Body:

The human body consists of typically 80% water. A disease such as, for example, Corona, is mainly caused by droplet infection. Here, the virus infects the body by mucosa via mouth, nose or eyes.

### Centralized Vaccination Station:

The water comprising the therein implemented information may also be bottled into bottles or other types of containers at a centralized vaccination station such to make that water available to individuals such as residents or patients.

### Cleaning of Water Pipes:

Due to the customary chlorination and a rinsing procedure, the water pipes may be cleaned from the water comprising the therein implemented information.

### Concerning Embodiment 3

The vaccine is bottled into bottles or other types of containers at a centralized location such to make it available to drugstore, pharmacies, doctors, etc. or such that it can be distributed by post.

### Further Application Possibilities of the Water of the Present Invention at Water Supply Companies:

less deposit of limescale in the piping system
less suspended solids
less predisposed for germs, etc. Reduced susceptibility to bacterial growth etc. Hence, also less chlorination or other treatment methods such as ozonation or blue-light therapy etc.

### Further Application Possibilities of the Water of the Present Invention at Sewage Plants:

improved coagulation, as the case may be, without addition of the customary chemical products
less chemical products in the ecological cycle as a whole
less chemicals in/on the agricultural fields. Farmers take, as customary, solid material from sewage plants and distribute that material on the agricultural fields. However, in this solid material are substances, some of which are neither good for agriculture, nor for groundwater, and especially not for food. For this reason, the amount of this material is limited, because the problem is known.

### Supplement to the Above-Described Treatment of Water

Referring now to FIG. 2, an inoculation my means of a dedicated water pipeline system of above-described Type B, shown in the figure, transfers the vaccine either to buildings (such as offices, domestic homes, etc.) or to a dedicated place of collection, such as stations and/or cabins. A main water pipeline system is arranged in substantially a ring shape. This main water pipeline system is also called vaccination ring. A house is connected to the main water pipeline system via a barrier with a Y-pipe included in the main water pipeline system. The barrier opens temporary or automatically. It automatically opens for a certain time and then automatically closes again. The house can be seen as an abstraction without backflow to the vaccination ring. Another water pipeline is connected to the main water pipeline system with two Y pipes included in the main water pipeline system, the water pipeline connecting a settlement, a single-family house and a doctor. Another water pipeline is connected via barriers to the main waltz pipeline system with two Y-pipes included in the main water pipeline system, the water pipeline connecting a hospital. Another water pipeline is connected via barriers to the main water pipeline system with two Y-pipes included in the main water pipeline system, the water pipeline connecting a supply/abstraction of vaccine and one or more pumps. The main water pipeline system further comprises multiple fixed installation for vaccination. A fixed installation for vaccination may for example be a cabin. The main water pipeline system further comprises an emergency station cabin.

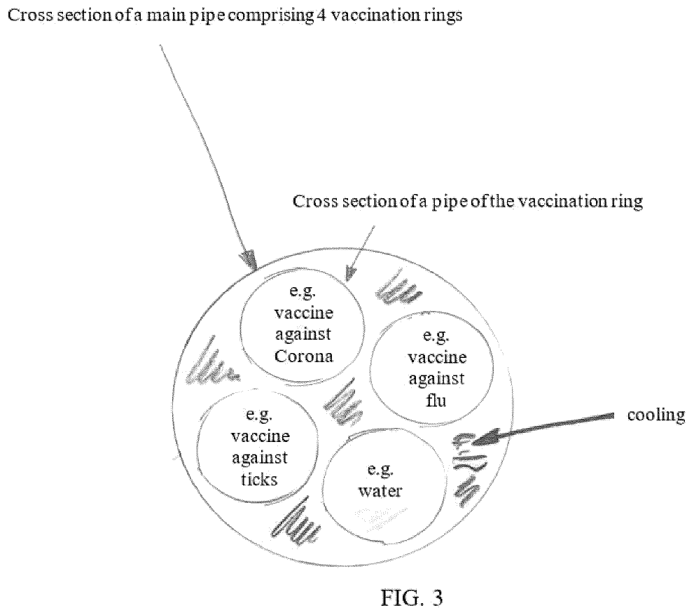

Referring now to FIG. 3 showing a schematic, cross-sectional view of a refrigerated carrier line including four different vaccination rings. A main pipe comprises four pipes of respective vaccination rings. Within the main pipe there is provided a cooling that surrounds the pipes of the vaccination rings. A first vaccination ring comprises e.g. a vaccine against Corona. One vaccination ring comprises e.g. a vaccine against Corona. A second vaccination ring comprises e.g. a vaccine against flu. A third vaccination ring comprises erg, water. A fourth vaccination ring comprises e.g. a vaccine against ticks.

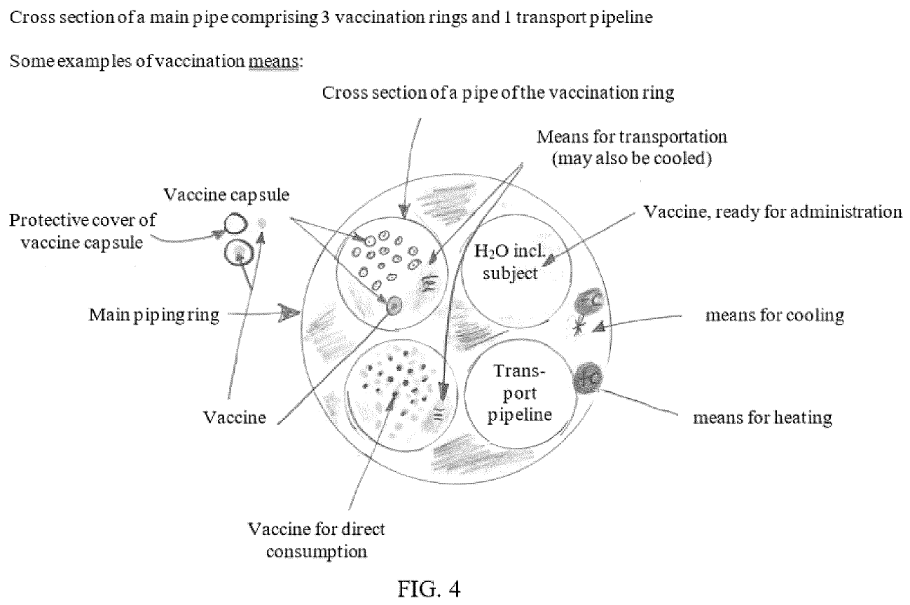

Referring now to FIG. 4 showing a schematic, cross-sectional view of a refrigerated carrier line enclosing four tubes, three of which are filled with different vaccinations and one is used as a transport tube. Within the main piping ring there is provided a means for cooling and a means for heating. The main piping ring comprises four tubes (pipes of vaccination ring). A first vaccination ring comprises vaccine capsules and means for transportation (which may also be cooled). A vaccine capsule comprises vaccine surrounded by a protective cover. A second vaccination ring comprises vaccine for direct consumption and means for transportation (which may also be cooled). A third vaccination ring comprises H₂O incl. a subject, e.g. a vaccine ready for administration. Such types of carrier lines may be used in dedicated water pipeline systems of Type B as schematically shown in FIG. 2.

The vaccine can be mixed directly in liquid form in the water, or in a kind of capsule, such as a bead or gelcapsule, etc., which is transported to the recipient only by the water, by compressed air or by another medium, which may be more viscous.

A viscous medium would prevent possible damage to the capsule, but would also slow the flow rate.

This transport medium can additionally have a cooling function, but heating for other products is also possible.

### Inoculation/Vaccination Pipelines (for the Transportation of Medical Substances):

As shown in FIG. 2 above, a vaccination pipeline can be crated, preferably as a ring pipeline (vaccination ring circuit/a closed circuit), in which only the vaccine is transported and so without water for domestic use. The vaccine nuns thereby always in the circle and can be taken at any time, unrestricted.

Inoculation/Vaccination Pipelines (Also Called Inoculation Rings), Transport Pipelines (Fir Different Substances) and Main Pipeline Rings (Combine Inoculation Rings and Transport Rings), the Branches of which go Directly to the Apartment Buildings, to the Apartments Itself, to Settlements, to Doctors, Hospitals, Inoculation Centers Etc.
Inoculation rings, transport pipelines/rings, main pipeline rings;
Pipelines that are fed directly to the apartment buildings and, if necessary, end there (possibly by automatically connecting the vaccination ring for a certain period of time and then automatically shutting it off) or are fed back into the original vaccination ring with a return pipeline (continuing vaccination ring), so that the vaccination ring always remains a closed vaccination ring or circuit, despite one or more branches that supply housing estates, housing complexes, doctors' surgeries, hospitals, etc.
But also normal water pipes, which can be used for direct inoculation or transport of capsules containing vaccines.
One or more emergency stations or cabins are provided for each housing estate;
Pipelines that extend directly into the apartments and single-family houses (e.g. at the time of construction of new buildings, the pipelines can be immediately incorporated into the building's infrastructure).

### Inoculation Through the Customary/Existing Water Pipe:

by dissolved vaccine;
through a vaccine capsule (vaccine in the capsule) or vaccine pearls (whole pearl is the vaccine) etc.;
vaccine capsules, the contents of which are not taken orally but are injected in the normal way.

These vaccine capsules, which are sprayed normally, would then only reach doctors and hospitals or vaccination centers via vaccination pipelines. The pipes would be laid to or opened only for these buildings, if necessary. With a distribution over the normal water pipe to all citizens, one would install, if necessary, before possible line obstacles like for example a water filter etc., in the respective houses, house plants, housing complexes a collecting tank before it, in order to remove the capsules before or to bridge this obstacle simply with a possibly switchable bypass line.

Emergency Stations/Cabins and their Sterilization for Delivery of Vaccination Etc.:

The Emergency Station/Cabin is located at or near the main pipeline ring, which is equipped with vaccination rings and transport pipelines if necessary.

An Emergency Station/Cabin, in which the patient collects his vaccination, among other things, is automatically disinfected. If necessary, the air in the room is sucked away and cleaned so that there are no more viruses in the air. In addition, the cabin and all contact surfaces can be sterilized by a short UV radiation between each patient. This Emergency Station/Cabin can be of a closed design, but also of an open design. No doctor's visit is necessary, which reduces the risk of being infected, no waiting periods, smaller costs etc.

### The Emergency Stations/Cabins

These are aid stations where the person seeking help can get everything necessary, such as water (cold and hot), gasoline, diesel, compressed air, blood (can be requested through the pipeline in a transport capsule), medical oxygen, medicines, insulin, protection from the weather, sandstorm, hail, hurricane, heat etc.

Items and medical equipment such as: Defibrillator, first aid kit, fire extinguisher, blanket etc. are located in the cabin or station.

Among other things, but also: vaccinations can be distributed here and vaccinated automatically.

Communication equipment of these emergency stations/cabins: telephone, satellite telephone, radio, Internet, W LAN station for Internet reception, power connection. USB adapter, connections of any kind, camera monitored, etc. Depending on the equipment and infrastructure, everything can be present or only parts of it.

Possibility of communication in the emergency station/cabin: emergency button, call to the fire department, police etc., contact to the doctor, avatar to help with all medical questions, who gives instructions if necessary, among other things, with the automatic vaccination etc.

Identification of the person seeking help or being helped: ID card, bank card, credit card, cell phone APP, QR-code, face recognition, voice recognition, hand or finger recognition, iris scanner, voice recognition etc. In a preferred embodiment, the data retrievable from such identification may be stored.

### Help Mode:

If a person is in distress, this person can talk to the cabin/station of the invention. No need to call a doctor for this (and no contact). A program including instructions which are read and executed by a CPU causes written or vocal instructions to be displayed/broadcast enabling the asking of questions about the distress and suffering to aid in providing assistance. If necessary, the program includes instructions enabling an avatar to communicate with the user and is programmed with instructions to automatically send an emergency call, if necessary, via radio/satellite, if no telephone and Internet connection is available, so that help comes. Thanks to optional translation module allowing for language selection, n, there are no language barriers.

### The Emergency Stations

This is an open aid station that provides a roof and sidewalls, if necessary, but is not completely enclosed and therefore not heated or air conditioned like the Emergency Cabin. Here, if necessary, you get everything u in the cabin, but the weather protection is only limited.

### Several Centralized Aid Decentralized Pick-Up Points (Emergency Cabins/Emergency Stations) in a City, Registration of the Person, Vaccination Card, Vaccination Certificate:

At these places the inhabitants of a city receive e.g. their vaccination dose in cue of a pandemic etc. If necessary, they can receive than without medical personnel, or medical personnel will vaccinate them.

There is also the possibility of automatic vaccination by a robot, automat or by positioning the arm or palm of an individual etc. over a certain opening, through which the vaccine is then introduced into the body. Vaccination can be completely automatic and patients are vaccinated without staff.

Also, an automatic registration of the person can take place at the same time, their data in a central computer, such as name, address, vaccine, date, station, blood group, diseases, etc. are stored. These vaccinations can thus be verified and can be deposited or stored in a separate personal file as a kind of vaccination passport. AU vaccinations, etc. can be registered here and, if necessary, viewed by the respective authorities, patients, etc. It would not be necessary to carry a vaccination document (cell phone app, website, etc.).

It may also be possible to speak directly to a doctor, staff member, avatar, etc. via a screen (by visual and/or audible communication).

Also, the connection option with this emergency station/cabin and the own cell phone is possible. Thus, among other things, the discretion towards third parties is guaranteed. The patient or customer connects his cell phone with the cabin or station and has direct contact with all its functions and possibilities, if necessary, via a separate app.

These emergency stations/cabins with their vaccination lines etc. can also be used, for example, for the annual flu vaccination or tick vaccination. A possible consultation, whether completely automated (avatar or robot etc.) or as a direct image and sound transmission with the respective doctor etc., is possible. Language selection and automatic translation function possible (via buttons, touch function, speech recognition etc.). Also, the personal conversation with the doctor etc., if necessary, with picture and sound, can be translated automatically into the respective language. Thus, there are no language barriers. Vacationers or foreigners can get their vaccinations etc. in different countries without visiting a doctor.

### Personal Identification/Vaccination Via Medical Card, Credit Card (Vaccination Tourism):

Face and voice recognition, palm print, etc. or via health card, credit card, bank card, coding system, QR code, etc.

Persons who do not have health insurance or are on vacation in the respective country can, if necessary, have the vaccination administered with your health card, bank card or credit card, facial recognition, etc. This vaccination would then be in this case possibly chargeable.

The vaccination to the respective person is registered in a system so that it can be used as a proof for a possible freedom of travel etc. all over the world.

For this, there would be the possibility of a cross-country registration system, which automatically leads each vaccinated in a database and each country or border can access this database. The respective vaccination can be downloaded as a proof on the cell phone (via their website or personal bar code, coding system, etc.).

Also, different vaccines can be administered by the day (useful in a vaccination ring/line circuit) so that the population can choose which vaccine they want to be vaccinated with and may have to pay extra than for the standard vaccine.

For this purpose, a website or an APP serves u an information platform for the fixed vaccination sites at the vaccination ring and the respective vaccination administered on the respective day at the respective site (second or third vaccination ring/closed vaccination administration system).

Future Legitimation ID Card, Bank Card, Health Card, Etc. Only Via Personal Markers Such as:
Face recognition
Speech recognition
Password request, if necessary spoken sentence, thereby speech recognition
Iris recognition
Handscan
Fingerscan
Breath
Blood or DNA

Through this query, in some cases only 2-3 of these markers are sufficient, each person can be clearly assigned. An ID card or a credit and health card will then be a thing of the past. All data can be stored centrally.

These documents are not necessary and can be falsified.

In a preferred embodiment, the data retrievable from such identification may be stored.

### Mobile Inoculation Stations

Mobile inoculation stations which are set up e.g. as mobile cabin (closed design), mobile station (open design), as car trailer, mobile tent, etc.
Such mobile stations are preferably located at collection points, at the ring pipeline or in its proximity. Mobile stations or cabins etc., which inoculate just in the case of pandemic automatically or by appropriate personnel.
For this purpose, there are several pick-up points on the ring pipeline to which the mobile vaccination station or cabin etc. can be directly connected. At these pick-up points, a mobile station may receive everything that a fixed station also has.
Such a mobile inoculation station can supplement permanently installed inoculation stations or completely replace them, so that there are only pick-up points on the ring pipeline which are approached by mobile inoculation stations, cabins, etc. or set up there and connected to the system.

### Advantages of a Vaccination Ring and the Vaccination Capsule:

This pipeline is used, for example, only for the vaccine. Either in direct liquid form, or as a capsule, etc., containing the vaccine. A vaccine capsule that you chew in your mouth (if necessary) and swallow the vaccine. If necessary, the capsule can be eaten or broken releasing its contents and then spit out.

It can also be a capsule containing the vaccine, which can be received at various central points, not only by the end user, but also by medical personnel, who may administer the vaccine to the patient by syringe, as usual. Injection guns can also be distributed to the population or be part of the first-aid kit, so that every citizen can administer the injection/vaccination himself in the event of a pandemic. Normal syringe and needle are already in a good first aid kit.

In addition, the whole capsule including the outer shell can be the vaccine (vaccine pearl).

Vaccine capsules as well as vaccine pearls ensure, among other things, the exact dosage of a drug or vaccine, etc.

At one or more points (unlimited number) on the ring line, unused vaccine etc. can be removed, or new vaccine etc. added.

An injection ring/ring pipeline does not have to be a ring in its form, it can be routed differently. Furthermore, pipelines can be attached to this ring to the inside or outside to better compensate for local conditions. It is important that these pipelines lead back to the injection ring/ring pipeline so that the closed circuit is guaranteed.

A vaccination pipeline that only leads to a house or multi-family dwelling and not back into the vaccination circuit is only automatically opened for a short period of time and automatically closed again. Only until sufficient vaccine has arrived at the property, etc.

This handling of automatically connecting and disconnecting another circuit to doctors, hospitals, the general public, etc. will be necessary mainly in a pandemic, etc. In normal operation, therefore, only the normal small circuit (vaccination circuit, main pipeline ring, transport power) runs through the Emergency Cabins, Emergency Stations, to ensure this supply, backup and use.

Form of the Inoculant:
Liquid form as concentrate or mixed ready for use
As a vaccination pearl for direct consumption
In a transport capsule (vaccine is in this capsule)
Transport container with one or more inoculation vials etc.
As a gel
As a powder
As a gas
As a capsule for usual injection by personnel
As a capsule etc. for injection by automatic machine
Encoded water or other encoded medium or product
etc.

Other Possible Uses of these Emergency Stations/Cabins:
Army, Bundeswehr etc. among others as a mobile version
Cities, villages etc.
Camps, such as refugee camps, etc.
Other planets/new settlements
etc.

Some Products that can be Transported, for Example, in the Injection Pipelines, Especially in the Ease of Multiple Injection Pipelines (without Flushing Process):
Vaccines
Medication
Transport capsules for consumption
Transport capsules for manual opening
Vaccination pearl
Antidotes/antidotes
Water
Gases
Fuels
Food
Blood
etc.
   Subdivides of Pipeline Rings into Main Pipeline Ring. Transport Pipeline and Injection Ring:
Inoculation ring: a ring pipeline, this can run in a pipeline with a larger diameter (main pipeline ring), e.g. to achieve cooling or heating.
Several vaccination rings, possibly with different vaccines etc., can be routed in one main pipeline ring and cooled or heated if necessary. Vaccines, antidotes etc. the vaccination rings take over the transport of sensitive substances such as blood and other medications etc.
Transport pipeline: A transport pipeline is used to transport and deliver any other substance, such as gasoline, gas, water cold/hot, food, etc.
Main pipeline rings can be operated at different temperatures. One main pipeline ring is hated, e.g. to 50 degrees Celsius, and another is cooled to zero degrees Celsius. This allows different substances to be transported.
The number of main pipeline rings may vary, as well as the number of inoculation rings and transport pipelines, possibly depending on an underline of the substances to be transported (drugs, vaccines, water, fuels, etc.). Also, the diameters of a main pipeline ring as well as an inoculation ring and the transport pipeline may be different.
   Main Line Rings and their Additional Cooling or Heating. If Necessary:

The diameter of a vaccination and transport pipeline can vary, e.g. one is 15 cm in diameter and another is only 5 cm in diameter. One can subject the medium transporting the vaccine, e.g. vaccine spheres/capsules etc. to additional cooling directly in the vaccination ring.

However, it is also possible to place the injection pipeline, which has a diameter of e.g. 15 cm, in a larger pipeline, a main pipeline ring, if necessary, also 5 such injection pipelines and these all run in a common, larger pipeline (main pipeline ring), which has a diameter of e.g. 100 cm. The space between these pipelines is then cooled or, if necessary, heated with the help of a cooling medium.

If, for example, a ready-to-drink vaccine is to be administered and the vaccine must not be diluted, the entire vaccine ring can thus be cooled from the outside through the larger pipeline and its cooling medium in the interstitial space accordingly.

If a product etc. has to be heated, the cooled line must of course be separated and insulated from the one to be heated (second main pipeline ring).

### Mobile Main Pipeline Ring, Mobile Transport Pipeline, Mobile Vaccination Ring Version, e.g. for Army, Armed Forces, Refugee Camp, Relief Organization, Etc.

Simple plug-in version of the pipelines or a roll-off line (including, but not limited to, gas, gasoline) etc., which can be quickly and easily laid if necessary.

Centralized (directly on the main ring) or decentralized feeding of the vaccine, etc.

Automatic administration of the vaccine, etc./medicine at multiple sites if necessary.

Via this Mobile Main Pipeline Ring, Transport Pipeline or Vaccination Ring can be Distributed, Among Others:
Vaccines
Blood
Medication
Water
Food
Oxygen and the like
Fuel
etc.

The vaccine etc. can be administered automatically or by medical personnel etc.

The inoculation ring can also be used as a distribution system for different needs.

Cooling may also be possible through the transport medium or through a second pipeline in which the injection ring is located. The gap between the injection pipeline/transport pipeline and the outer pipeline is then cooled with a cooling medium.

Multiple inoculation rings that transport or administer different substances are also possible.

The number of acceptance points, inoculation stations, etc. is unlimited.

The possibility of acceptance, etc. is possible as in the case of the fixed city.

### Other Possible Applications:

New Housing Complexes and Settlements, Also on Other Planets or Celestial Bodies, Such as Mars, Moon Etc.:
Vaccination rings
Medication and vaccinations
Oxygen (gases) etc.
Fuels
Heat transport
Water transport
Food
etc.

In an advantage, the invention allows for the transfer of beneficial information as well as beneficial substances through the water into living organisms which consume the water.

In another advantage, the invention allows for the treatment of living organisms en mass using pre-existing or dedicated piping as a distribution system.

In an advantage, the invention allows an administration of a vaccine without the need of a syringe in order to inject the vaccine, for example, into the bloodstream.

In an advantage, the distribution system of the invention avoids doctor visits and the associated infection risk, no waiting periods, lower costs etc.

In another advantage, authorities are provided with the ability to mete out beneficial treatments, in some cases using beneficial substances carried in the water lines, on an as needed basis, according to a treatment protocol that the authorities believe to be most effective.

In another advantage, a user to be treated is identified such that a treatment certificate can be issued on the spot at a place of need, such as at an airport.

### Surveillance of Sewage Water;

In the following, embodiments are described which provide for wastewater testing for markers, compositions, but also for viruses, such as coronaviruses and their mutations:

In wastewater, for example, the pathogen RNA (ribonucleic acid) is directly detectable as well as other markers detectable by probes and other equipment.

Investigations of the wastewater, such as at the sewage treatment plants, but also directly at the hotels, companies, workers' camps, restaurants, homes, etc.

Direct monitoring of these localities, and not only of wastewater treatment plants, is new, whether by individual sampling, by personnel or by an automatic system.

By direct examination of important localities, e.g. hotels, the catering trade enterprise can be kept upright, because all guests are examined 24 h/day for a possible infection etc.

Direct testing of wastewater, such as from hotels, can determine within 24-72 hrs whether or not a hotel is harboring a diseased individual.

Through the direct probe monitoring of the individual toilets, can be determined in real time thus IMMEDIATELY (about 5 minutes) whether a guest is sick in the hotel.

Example: A guest is on the toilet: The system measures the stool and urine and determine e.g., the guest is Corona-positive, then the system would be able to report this positive measurement automatically to the hotel management and third parties. The hotel could react immediately, you would know exactly the room, date and time and could assign and isolate this person immediately. By a person registration of the toilet e.g. by fingerprint, language, QR code etc. can be assigned additionally each person also if this I e.g. on a guest toilet e.g. in a restaurant is.

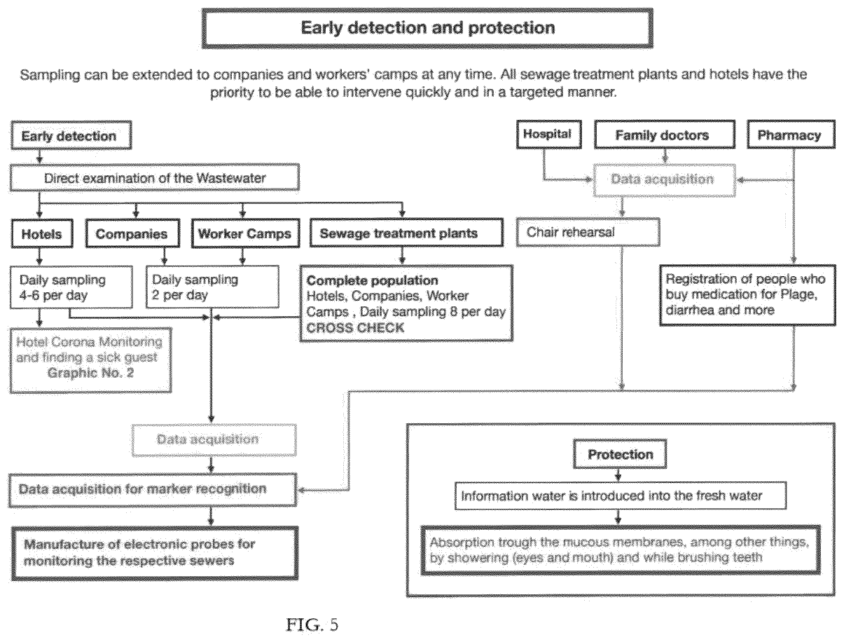

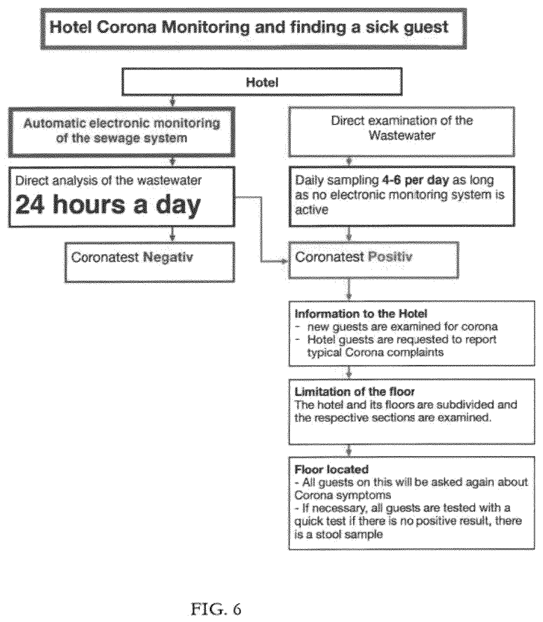

FIG. 5 schematically shows a process of early detection and protection according to an embodiment. The process of early detection comprises direct examination of the wastewater. In the embodiment, this direct examination of the wastewater is applied in hotels, companies, worker camps, and sewage treatment plants. In a hotel, for example, a daily sampling of 4-6 times per day is applied. In companies and worker camps, for example, a daily sampling of 2 times per day is applied. Via the sewage treatment plants a monitoring of the complete population (hotels, companies, worker camps) may be applied as a cross check on a basis of a daily sampling of 8 times per day. Data acquisition collects the information from the daily sampling of the hotels, the daily sampling of the companies, the daily sampling of the worker camps, and the daily sampling of the sewage treatment plants. Still further, data is acquired from hospitals, family doctors, and pharmacy. Data acquired data is provided to chair rehearsal. Further information is obtained by registration of people who buy medication, e.g. for Plage, diarrhe and more at pharmacy. A data acquisition for marker recognition is performed based on the data acquired from the daily sampling of hotels, companies, worker camps, and sewage treatment plants, and based on the data acquired from the hospital, family doctors, and pharmacy, and based on the information from the registration of people who by medication. Based on the information obtained from data acquisition for marker recognition, a manufacturing of electronic probes for monitoring the respective sewers is performed. Still further, based on the daily sampling of hotels, a hotel Corona monitoring and a finding of sick guests as performed. This hotel Corona monitoring and a finding of sick guests is described in more detail with regard to FIG. 6 below. FIG. 5 also shows a process of protection. Information water is introduced into the fresh water. This may for example be performed as set out in the embodiments described above in more detail. The information water is absorbed through the mucous membranes, among other things, by showering (eyes and mouth) and while brushing teeth. More detailed aspects of this detection and protection process are described below.

It should be noted that sampling can be extended to companies and waiters camps at any time. All sewage treatment plants and hotels have the priority to be able to intervene quickly and in a targeted manner.

FIG. 6 schematically shows a process of hotel Corona monitoring and finding a sick guest according to an embodiment. In a hotel, an automatic electronic monitoring of the sewage system is applied. For example, direct analysis of the wastewater is performed 24 hours a day, comprising performing a Corona test. Still Rather, a direct examination of the wastewater is performed. For example, a daily sampling of 4-6 times per day is performed as long as no electronic monitoring system is active. The examination of the wastewater comprises a Corona test. If either the Corolla test performed by automatic electronic monitoring of the sewage system or the Corona test performed by direct examination of the wastewater is positive, respective information is provided to the hotel. This may result in examining new guests for Corona or requesting hotel guests to report typical Corona complaints. A limitation to floors may be applied. To this end, the hotel and its floors are subdivided into sections (acres) and the respective sections are examined. Once an affected floor has been located, all guests on this floor will be asked again about Corona symptoms. If necessary, all guests are tested with a quick test; if there is no positive result, there is a stool sample analyzed. More detailed aspects of this monitoring process are described below.

### Finding a Sick Person Through the Sewage:

Checking the waste water of a hotel.
How to find the floor or the infected?

On one day, between 4-6 samples are taken from one hotel. Thus, after about 72 hours, up to 18 wastewater samples were taken from the same hotel and analyzed.

The number of samples may vary.

If there are no Corona viruses in the 18 wastewater samples, the hotel is 98% certain not to be hosting a guest who has contracted Corona.

If corona virus is detected in a sample on a subsequent day, there is a 60% probability that it originates from a new guest for whom the PCR test was unsuccessful. This would also confirm the type of corona virus found or help to determine whether it is a mutation that can no longer be detected with a PCR test.

Thus, the first step would be to test all newly arrived guests for corona infection. Additionally, stool samples of the guests could be examined, since a stool sample is more reliable than a PCR test in the examination for coronaviruses.

The hotel would also be informed that we found coronaviruses in their hotel and the hotel may initiate tightened security measures.

Depending on the sewage line in the hotel, the hotel could be divided into three areas, for example, a hotel has 60 floors, so you would draw 2 samples in the hotel.

1st sample: on the 40th floor, thus the floors 60-40 would be checked.

2nd sample: on the 20th floor, so floors 39-20 would be checked.

If there is no positive sample, the infected person must be in the first 19 floors.

In this way, you would continue to narrow down these 19 floors until you found the floor. Always from top to bottom, since it is a down pipe. If there is no direct access to the downpipe, one would remove the nearest toilet in a hotel room etc. at the downpipe on the respective floor in order to get to the downpipe via the access.

Arriving at the correct floor, one would unceremoniously check these guests directly into account who was the most recent new arrival and who may have symptoms.

The hotel would also ask the guests' symptom query to all hotel guests as soon as a positive corona finding was detected in their wastewater.

Finding an infected hotel guest will be 90% detectable within 48 hrs.

### Sampling:

by one person
Automatic sampling, special device
Probes and other measurement electronics
Camera
Odor Sensing
etc.

### Attaching the Automatic Sensor Measurement or Electronic Monitoring:

In the case of a hotel, if necessary, on each floor at the wastewater downpipe or line. Thus the allocation of the floor.
In each room directly to the toilet or sewage pipe (Thus, the direct assignment of the room and its occupants). The Monitoring of the floor could thus be eliminated.
Merging wastewater pipe of a hotel, inflow into the sewer system or outflow from the hotel. Thus monitoring the entire hotel.
Toilets that include the complete system incl. person recognition. Direct assignment of the person no matter whether hotel guest or only guest of the restaurant or bar etc.
Everywhere where monitoring makes sense.

### Measurements Directly at the Toilet in the Hotel Room:

It is also possible to retrofit each toilet (e.g. in a hotel) or its pipe with the respective sensor technology.

In this cue, the sick person or the hotel room could be assigned immediately.

Either via a transmission cable or via radio, W-LAN etc., data can be transmitted directly to the hotel, the authority or third or fourth parties etc.

Due to the already wasting, distinctive camera surveillance in every hotel, also the guest toilets in a hotel, restaurant etc. and their users can be assigned in the end.

### Medical Toilet to Check the Health Status and Check for Infection Such as Corona Virus:

Task:

Measurements of various parameters, such as:
Weight
Urine examination
Examination of the stool
Blood examination
Condition (per camera, among other things)
etc.

### Determinations of Diseases:

infections via viruses, puck as corona viruses and their mutations, etc.
Gastrointestinal infections
Insulin
Fat
Digestive function
Pregnancy
Blood in stool or urine
Health status
Fluid deficiency
etc.

### Personalization of the Toilet:

User recognition of toilet by e.g. voice recognition, weight with demand of toilet (voice), fax recognition, user input, fingerprint, Personal QR-Code etc.
Data transmission by wire, W-LAN, radio etc.

### Function and Procedure:

The toilet is fed with user-relevant data, such as name; age; gender, weight, which the toilet additionally measures; any diseases (such as sugar) etc.
The user sits down on the toilet and, for example, via the weight, the toilet asks additionally for his name, if necessary. When doing business on the toilet, examinations are automatically performed. The urine, as well as the stool, is automatically examined by probes, odor probes and other detection.

### Output of the Data:

As a rule, the examination via probes etc. does not require 5 minutes. If the examination is not completed and the user is finished, the toilet flushes automatically only after the examination is completed, thus with a time delay.

The results of the examination are then transmitted and stored, e.g. via W-LAN transmission, on the cell phone, computer, pad or a screen specially designed for the toilet. If necessary, this data can also be transmitted to third parties and fourth parties etc.

### Transfer to Primary Care Physician, Care to Third Party, Etc.:

These toilets find application e.g. in hospitals, nursing homes, assisted living, hotels, companies (that monitor their employees health), private households, cruise ships, airplanes etc.

In assisted living, for example, this data can be automatically transmitted directly to the primary care physician.

The data can also be transferred to and stored on an electronic medical card.

Still further, the data may be transmitted via a telematic infrastructure (TI) to a third party. The telematics infrastructure (TI) is intended to enable secure networking of medical care within a country. Connecting medical practices and hospitals ensures that medical data and documents can be sent to the treating physicians quickly and easily in a secure manner. The telematics infrastructure (TI) is a “data highway” of the healthcare system which is designed to enable fast and secure communication between doctors, psychotherapists, hospitals and others. Medical information needed for the treatment of patients is thus available more quickly and easily.

### Automatic Diagnosis

The toilet could automatically draw conclusions about the user's state of health on the basis of the findings and, if necessary, advise different food, vitamins, a visit to the doctor, etc. The user would then be able to choose the right food. Even the selection of possible vitamins and foods could be suggested to the user by the toilet or its program, if necessary, recommend the doctor or gym in the area, etc.

Thus, complete coverage of the user with all his concerns would be achieved. Marketing for companies, doctors, products and services etc. would be covered by this toilet.

Automatic Cleaning and Disinfection of the Toilet May Comprise:
Automatic flushing
Disinfectant
UV light irradiation (germ-free). Thus no waste water pollution
etc.

### Medical APP:

The embodiments also provide a cell phone app on which users register their disease or current symptoms, e.g. by entering the personal data or a personal QR code that has all the desired personal data in it.

Also the examined data of the medical toilets could flow in here. This can be used to determine whether a focus of disease, a virus, etc. is spreading in a village, a city, a country, etc.

Hospitals and general practitioners enter markers into a computer system, e.g. fever, diarrhea, etc. Patients are recorded or registered here with their symptoms.

Pharmacies register the sale of e.g. antipyretic, antidiarrheal. If necessary, the customer only gives his QR code and the system records all personal data.

This provides an up-to-date picture of the health status of a city, country, etc.

A unique feature of this app is that it collects, evaluates and forwards data from the toilets.

### Computer Program & App:

By storing data possibly also by the medical toilets, the health status of the population can be shown. Flu viruses, diseases and infections, such as corona diseases, can be graphically displayed using a city or country map.

Water Pipes for Dual Use, Etc.:

Capsule, vaccines etc. (all substances as already listed in the patent specification . . . ) which are transported into existing water mains and if necessary normal water pipes.

In existing water pipes, a new pipe is laid, which may lead several other pipes in itself.

Advantage: An existing pipeline system can be used partly on the complete length to the destination or partly. The usual water main has a diameter of 130 cm. A line of e.g. 20 cm can be laid into this main line without any problems. Thus, one saves the laying costs and saves time.

Capsules that can be Taken from Water Meters:
Capsules transported through the pipeline system are taken at modified existing old water meters.
New water clocks specifically for the withdrawal for such capsules
Removal possibility on a special piece of pipe which has been inserted.

It should be appreciated that the particular implementations shown and herein described are representative of the invention and its beat mode and are not intended to limit the scope of the present invention in any way.

It should be appreciated that many applications of the present invention may be formulated. One skilled in the art will appreciate that the network may include any system for exchanging data, such as, for example, the Internet, an intranet, an extranet, WAN, LAN, wireless network, satellite communications, and/or the like. It is noted that the network may be implemented as other types of networks, such as an interactive television network. The users may interact with the system via any input device such as a keyboard, mouse, kiosk, personal digital assistant, handheld computer, cellular phone and/or the like. Moreover, the system contemplates the use, sale and/or distribution of any goods, services or information having similar functionality described herein.

The computing units mentioned herein may be connected with each other via a data communication network. The network may be a public network and assumed to be insecure and open to eavesdroppers, or may also be secure. In one exemplary implementation, the network may be embodied as the Internet. In this context, the computers may or may not be connected to the Internet at all times.

As will be appreciated by skilled artisans, the present invention may be embodied as a system, a device, an apparatus, or a method.

The present invention is described herein with reference to block diagrams, devices, components, and modules, according to various aspects of the invention. It will be understood that each functional block of the blocks diagrams, and combinations of functional blocks in the block diagrams, can be implemented by computer program instructions which may be loaded onto a general purpose computer, special purpose computer, or other programmable data processing apparatus to produce a machine, such that the instructions which execute on the computer or other programmable data processing apparatus create enable the functionality specified in the block diagrams.

Accordingly, the block diagram illustrations support combinations of means for performing the specified functions, combinations of steps for performing the specified functions, and program instruction means for performing the specified functions. Each fractional block of the block diagrams and flowchart illustrations, and combinations of functional blocks in the block diagrams, may be implemented by either special purpose hardware-based computer systems which perform the specified functions or steps, or suitable combinations thereof.

Moreover, the system contemplates the use, sale and/or distribution of any goods, services or information having similar functionality described herein.

The specification and figures should be considered in an illustrative manner, rather than a restrictive one and all modifications described herein are intended to be included within the scope of the invention claimed. Accordingly, the scope of the invention should be determined by the appended claims (as they currently exist or as later amended or added, and their legal equivalents) rather than by merely the examples described above. Steps recited in any method or process claims, unless otherwise expressly stated, may be executed in any order and are not limited to the specific order presented in any claim. Further, the elements and/or components recited in apparatus claims may be assembled or otherwise functionally configured in a variety of permutations to produce substantially the same result as the present invention. Consequently, the invention should not be interpreted as being limited to the specific configuration recited in the claims.

Benefits, other advantages and solutions mentioned herein are not to be construed as critical, required or essential features or components of any or all the claims.

As used herein, the terms “comprises”, “comprising”, or variations thereof, are intended to refer to a non-exclusive listing of elements, such that any apparatus, process, method, article, or composition of the invention that comprises a list of elements, that does not include only those elements recited, but may also include other elements such as those described in the instant specification. Unless otherwise explicitly stated, the use of the term “consisting” or “consisting of” or consisting essentially of is not intended to limit the scope of the invention to the enumerated elements named thereafter, unless otherwise indicated. Other combinations and/or modifications of the above-described elements, materials or structures used in the practice of the present invention may be varied or adapted by the skilled artisan to other designs without departing from the general principles of the invention.

The patents and articles mentioned above are hereby incorporated by reference herein, unless otherwise noted, to the extent that the same are not inconsistent with this disclosure.

Other characteristics and modes of execution of the invention are described in the appended claims.

Further, the invention should be considered as comprising all possible combinations of every feature described in the instant specification, appended claims, and/or drawing figures which may be considered new, inventive and industrially applicable.

Additional features and functionality of the invention are described in the claims appended hereto and/or in the abstract. Such claims and/or abstract are hereby incorporated in their entirety by reference thereto in this specification and should be considered as part of the application as filed.

Multiple variations and modifications are possible in the embodiments of the invention described here. Although certain illustrative embodiments of the invention have been shown and described here, a wide range of changes, modifications, and substitutions is contemplated in the foregoing disclosure. While the above description contains many specific details, these should not be construed as limitations on the scope of the invention, but rather exemplify one or another preferred embodiment thereof. In some instances, some features of the present invention may be employed without a corresponding use of the other features. Accordingly, it is appropriate that the foregoing description be construed broadly and understood as being illustrative only, the spirit and scope of the invention being limited only by the claims which ultimately issue in this application.

### LITERATURE

It has been proven that certain water can more easily enter the cell structure and have an effect on them.

The following documents are incorporated by reference herein and relied upon in support of the above point, among other points:
Roland Plocher (Praktische Anwendung, TV—Ausstrahlungen, Presse etc.)
Wilhelm Reich (Krebsforschung, Zellen etc.) Buchtitel: Die Entdeckung des Orgons—Der Krebs; 5. Auflage 2018, The Cancer Biopathy 1948, Orgone Institute Press, Inc.; 1971, 1994 by Verlag Kiepenheuer & Witsch, Köln; ISBN: 978-3-462-02378-7
Wilhelm Reich, Buchtitel: Die Entdeckung des Orgons—Die Funktion des Orgasm us; 11. Auflage 2019; THE FUNCTION OF THE ORGASM (The Discovery of the Orgone, Vol. I, 1942, 1948, 1961 Orgone Institute Press Inc., 1969, 1987 by Verlag Kiepenheuer & Witsch GmbH & Co. KG Köln; ISBN: 978-3-462-01825-7
Janes DeMeo, Buchtitel: Das Orgonakkumulator Handbuch; 1. Auflage 2015; The Orgone Accumulator Handbook: Wilhelm Reich's Life-Energy Discoveries and Healing Tools for the 21^{st} century, with Construction Plans; Verlag Natural Energy Works, Ashland Oregon, USA; 1989, 1994, 2010 by James DeMeo; ISBN: 978-0989139039 0989139034
Bernd Serf, Buchtitel: Die Wiederentdeckung des Lebendigen: Erforschung der Lebensenergie; 1. überarbeitete Neuauflage 2019; 2003 Omega Verlag; 1996 Zweitausendeins, Frankfurt am Main; ISBN: 978-3-89845-636-4 durch Reich, Schauberger, Lakhovsky, Schmidt, Pincher Herbert and Knapp
Dieter Knapp, Forschungsinstitut für Radiästhie and Biophysik, Fürth Odenwald
Georges Lakhovsky, Buchtitel: Des Geheimnis des Lebens. Kosmische Wellen und vitale Schwingungen
Georges Lakhovsky, Buchtitel: Bioenergetische Schwingungen and Resonanz
Olaf Alexandersson, Buchtitel: Lebendes Moser
Paul F. M. Nogier, Buchtitel: Lehrbuch der Auriculotherapie
Paul Schmidt, Buchtitel: Schwingung and Heilung
“Kurzer Prozess im Wassercluster”, Rainer Scharf, https//www.pro-physik.de/nachrichten/kurzer-prozess-im-wassercluster
T Jahnke et al, Nature Physics (online Oct. 1, 2010), “Ultrafast energy transfer between water molecules”, http://dx.doi.org/10.1038/nphys1498

1-28. (canceled)

29. A method for monitoring of sewage water, comprising testing the sewage water, using water testing means, for particular infectious agents or contaminants, including detecting their type/nature/variety and/or their density of presence.

30. The method of claim 29, further comprising reporting detected infectious agents or contaminants to a central processor.

31. The method of claim 30, further comprising mapping, by the central processor, the detections on a map, thereby giving authorities an almost immediate, real-time indication of hot spots and regions likely to become super spreaders.

32. The method of claim 29, further comprising direct probe monitoring of individual toilets of a building.

33. The method of claim 29, further comprising determining in real time whether a guest in a hotel is sick.

34. The method of claim 29, further comprising divided a building into areas depending on the sewage line in the building and drawing samples within the areas of the hotel.

35. The method of claim 29, further comprising attaching automatic sensor measurement or electronic monitoring on each floor of a building at the wastewater downpipe or line.

36. The method of claim 29, further comprising attaching automatic sensor measurement or electronic monitoring in each room directly to the toilet or sewage pipe.

37. The method of claim 29, further comprising retrofitting a toilet or its pipe with the respective sensor technology for sewage monitoring.

38. The method of claim 29, further comprising transmitting the monitoring result and storing the result on a cell phone, computer, pad or a screen.

39. The method of claim 29, further comprising automatically drawing conclusions about a toilet user's state of health on the basis of the monitoring result.

40. The method of claim 29, further comprising advising, on the basis of the monitoring result, different food, vitamins, and/or a visit to the doctor to the user.

41. The method of claim 29, further comprising storing data related to the monitoring result by a medical toilet.

42. The method of claim 29, further comprising graphically displaying information that is based on the monitoring result using a city or country map.

43. The method of claim 29, further comprising a direct examination of the wastewater in a hotel, a company, a worker camps, or at a sewage treatment plant.

44. The method of claim 29, further comprising acquiring data from hospitals, family doctors, or pharmacy.

45. The method of claim 29, further comprising acquired data to chair rehearsal.

46. The method of claim 29, further comprising obtaining information from registration of people who buy medication.

47. The method of claim 29, further comprising a data acquisition for marker recognition, and performing, based on the information obtained from data acquisition for marker recognition, a manufacturing of electronic probes for monitoring respective sewers.

48. The method of claim 29, further comprising, based on daily sampling of hotels, performing a hotel Corona monitoring.

49. The method of claim 29, further comprising, based on daily sampling of hotels, finding of sick guests.

50. The method of claim 29, further comprising an automatic electronic monitoring of a sewage system.

51. The method of claim 29, further comprising a direct analysis of wastewater 24 hours a day.

52. The method of claim 29, further comprising a direct analysis of wastewater as long as no electronic monitoring system is active.

53. The method of claim 29, further comprising performing a Corona test.

54. The method of claim 51, wherein the direct examination of the wastewater comprises a Corona test.

55. The method of claim 29, further comprising, if either a Corona test performed by automatic electronic monitoring of the sewage system or a Corona test performed by direct examination of the wastewater is positive, providing respective information to a hotel.

56. The method of claim 52, wherein the direct examination of the wastewater comprises a Corona test.
